(19) United States
(12) Patent Application Publication
(10) Pub. No.: US 2025/0186204 A1
(43) Pub. Date: Jun. 12, 2025

(54) PROXIMAL LOADER ASSEMBLY FOR LOADING HEART VALVE INTO DELIVERY DEVICE

(71) Applicant: Boston Scientific Scimed, Inc., Maple Grove, MN (US)

(72) Inventors: Richard Kiely, Galway (IE); Tim O'Connor, Galway (IE); Pearse A. Coffey, Galway (IE); Neil O'Connor, Galway (IE)

(73) Assignee: Boston Scientific Scimed, Inc., Maple Grove, MN (US)

(21) Appl. No.: 18/977,521

(22) Filed: Dec. 11, 2024

Related U.S. Application Data
(60) Provisional application No. 63/609,094, filed on Dec. 12, 2023.

(51) Int. Cl. A61F 2/24

(52) U.S. Cl. CPC A61F 2/243

(57) ABSTRACT

A proximal loader may be used to facilitate loading a prosthetic valve into a catheter. A proximal load may include an elongate body releasably securable about a catheter, the elongate body including a first body half hingedly coupled with a second body half. Each of the first body half and the second body half includes an outer shell, a distal member segment disposed within the outer shell and defining half of a valve crimping region, and a proximal member segment slidingly disposed within the outer shell and defining half of a catheter accepting region.

### CROSS-REFERENCE TO RELATED APPLICATIONS

This application claims the benefit of priority of U.S. Provisional Application No. 63/609,094 filed Dec. 12, 2023, the entire disclosure of which is hereby incorporated by reference.

### TECHNICAL FIELD

The disclosure pertains to medical devices and more particularly to loader assemblies for loading a heart valve into a delivery system, and methods for using such medical devices.

### BACKGROUND

A wide variety of medical devices have been developed for medical use including, for example, artificial heart valves for repair or replacement of diseased heart valves. The artificial heart valve must be carefully loaded into a delivery system. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example may be found in a proximal loader. The proximal loader includes an elongate body releasably securable about a catheter, the elongate body including a first body half hingedly coupled with a second body half. Each of the first body half and the second body half include an outer shell, a distal member segment disposed within the outer shell and defining half of a valve crimping region, and a proximal member segment slidingly disposed within the outer shell and defining half of a catheter accepting region.

Alternatively or additionally, each distal member segment may be fixedly secured to the corresponding outer shell.

Alternatively or additionally, each distal member segment may include a distal insert that is fastened to the outer shell.

Alternatively or additionally, each distal member segment may be integrally molded as part of the corresponding outer shell.

Alternatively or additionally, each proximal member segment may be slidingly secured to the corresponding outer shell.

Alternatively or additionally, each of the first body half and the second body half may further include one or more biasing members extending between the proximal member segment and the corresponding outer shell.

Alternatively or additionally, the one or more biasing members may include springs.

Alternatively or additionally, each proximal member segment may include a proximal insert having two or more elongate mounting slots formed within the proximal insert, and the proximal insert may be slidingly secured to the corresponding outer shell via fasteners extending through each of the two or more elongate mounting slots and engaging in the corresponding outer shell.

Alternatively or additionally, the first body half and the second body half may each include a semicircular outer threaded surface adapted to threadedly engage a pusher cap that is used to advance the prosthetic valve proximally into the catheter.

Alternatively or additionally, each proximal member segment may further define a catheter engaging region that is adapted to secure the proximal member relative to the catheter.

Another example may be found in a proximal loader. The proximal loader includes an outer shell, a distal member disposed within the outer shell, the distal member defining a valve crimping region, and a proximal member slidingly disposed within the outer shell, the proximal member defining a catheter accepting region. The proximal member further defines a catheter engaging region that is adapted to secure the proximal member relative to the catheter. The proximal loader is adapted to be hingedly opened in order to secure the proximal loader about the catheter.

Alternatively or additionally, the outer shell may include a first shell half and a second shell half.

Alternatively or additionally, the distal member may include a first distal member segment disposed within the first shell half and a second distal member segment disposed within the second shell half.

Alternatively or additionally, the first distal member segment may be fixedly secured within the first shell half and the second distal member segment may be fixedly secured within the second shell half.

Alternatively or additionally, the proximal member may include a first proximal member segment slidingly secured within the first shell half and a second proximal member segment slidingly secured within the second shell half.

Alternatively or additionally, the first proximal member may be slidingly secured within the first shell half via one or more fasteners extending through the first proximal member and into the first shell half, and the second proximal member may be slidingly secured within the second shell half via one or more fasteners extending through the second proximal member and into the second shell half.

Alternatively or additionally, the proximal loader may further include one or more biasing members extending between the outer shell and the proximal member.

Another example may be found in a proximal loader. The proximal loader includes a first outer shell, a first distal member segment disposed within the first outer shell, a first proximal member segment slidingly disposed within the first outer shell, a first biasing member disposed between the first proximal member segment and the first outer shell, a second outer shell hingedly secured to the first outer shell, a second distal member segment disposed within the second outer shell, the first distal member and the second distal member together defining a valve crimping region, a second proximal member segment slidingly disposed within the second outer shell, the first proximal member segment and the second proximal member segment together defining a catheter accepting region, and a second biasing member disposed between the second proximal member segment and the second outer shell.

Alternatively or additionally, the first biasing member may bias the first proximal member segment towards the first distal member segment and the second biasing member may bias the second proximal member segment towards the second distal member segment.

Alternatively or additionally, the first proximal member segment and the second proximal member segment together may define a catheter engaging region that is adapted to secure a catheter extending between the first proximal member segment and the second proximal member segment against movement relative to the first proximal member segment and the second proximal member segment.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative replacement heart valve implant;

FIG. 2 is a schematic view of an implant delivery system usable with the replacement heart valve implant of FIG. 1;

FIG. 3 is a schematic view of an illustrative proximal loader and pusher cap that may be used in assisting to load the illustrative replacement heart valve implant of FIG. 1 into a delivery device;

FIG. 4 is a front view of a portion of the illustrative proximal loader of FIG. 2, including a distal member segment and a proximal member segment, with the proximal member segment shown in a relaxed configuration;

FIG. 5 is a front view of the portion of the illustrative proximal loader of FIG. 2, including a distal member segment and a proximal member segment, with the proximal member segment shown displaced from the relaxed configuration;

FIG. 6 is a front view of the illustrative proximal loader of FIG. 2, showing a step in using the illustrative proximal loader;

FIG. 6A is an enlarged view of a portion of FIG. 6;

FIG. 7 is a front view of the illustrative proximal loader of FIG. 2, showing a step in using the illustrative proximal loader;

FIG. 8 is a front view of the illustrative proximal loader of FIG. 2, showing a step in using the illustrative proximal loader;

FIG. 9 is a schematic view of the implant delivery system after loading the replacement heart valve implant of FIG. 1;

FIG. 10 is a front view of a portion of the illustrative proximal loader of FIG. 2, including an outer shell;

FIG. 11 is a back view of a distal insert that forms a distal member segment;

FIG. 12 is a back view of a proximal insert that forms a proximal member segment; and

FIG. 13 is a front view of a portion of an illustrative proximal loader in which the distal member segment is integrally formed within the outer shell.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

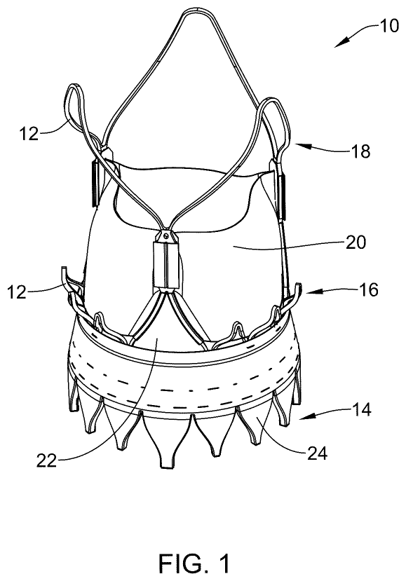

FIG. 1 is a perspective view of an illustrative replacement heart valve implant 10. It should be appreciated that the replacement heart valve implant 10 may be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

The replacement heart valve implant 10 may include an expandable framework 12 defining a central lumen. In some instances, the expandable framework 12 may have a substantially circular cross-section. In some instances, the expandable framework 12 may have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 may be configured to shift between a radially collapsed configuration and a radially expanded configuration. In some instances, the expandable framework 12 may be self-expanding. In some instances, the expandable framework 12 may be self-biased toward the radially expanded configuration. In some instances, the expandable framework 12 may be mechanically expandable. In some instances, the expandable framework 12 may be balloon expandable. Other configurations are also contemplated. In some instances, the expandable framework 12 may include and/or define a plurality of interstices (e.g., openings) through the expandable framework 12.

In some instances, the expandable framework 12 may define a lower crown 14 proximate an inflow end, an upper crown 16 proximate an outflow end, and a plurality of stabilization arches 18 extending downstream from the outflow end. In some instances, the lower crown 14 may be disposed at the inflow end. In some instances, the upper crown 16 may be disposed at the outflow end. In some instances, the expandable framework 12 may include a tubular wall defining the central lumen, the inflow end, the outflow end, the lower crown 14, and/or the upper crown 16. In some instances, the plurality of stabilization arches 18 may extend downstream of and/or away from the upper crown 16 in a direction opposite the lower crown 14. In some instances, the upper crown 16 may be disposed longitudinally and/or axially between the lower crown 14 and the plurality of stabilization arches 18.

In some instances, the replacement heart valve implant 10 may include a proximal portion and a distal portion. In some instances, orientation of the replacement heart valve implant 10 may be related to an implant delivery device and/or a direction of implantation relative to a target site. In some instances, the proximal portion may include the outflow end and/or the plurality of stabilization arches 18. In some instances, the proximal portion may include the upper crown 16 and/or the plurality of valve leaflets 20. In some instances, the distal portion may include the inflow end and/or the lower crown 14. Other configurations are also contemplated.

In some instances, the replacement heart valve implant 10 may include a plurality of valve leaflets 20 disposed within the central lumen. The plurality of valve leaflets 20 may be coupled, secured, and/or fixedly attached to the expandable framework 12. In some instances, the outflow end of the expandable framework 12 may include the plurality of stabilization arches 18 extending axially away from the plurality of valve leaflets 20 and/or from a commissure (or commissures) or an attachment point (or attachment points) of the plurality of valve leaflets 20 with the expandable framework 12.

In some instances, the replacement heart valve implant 10 may include an inner skirt 22 disposed on and/or extending along an inner surface of the expandable framework 12. In at least some instances, the inner skirt 22 may be fixedly attached to the expandable framework 12. The inner skirt 22 may direct fluid, such as blood, flowing through the replacement heart valve implant 10 toward the plurality of valve leaflets 20. In at least some instances, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some instances, the replacement heart valve implant 10 can include an outer skirt 24 disposed on and/or extending along an outer surface of the expandable framework 12. In some instances, the outer skirt 24 may be disposed at and/or adjacent the lower crown 14. In some instances, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some instances, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some instances, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated. The expandable framework 12 may include additional structure components that are not shown in FIG. 1. In some instances, some of the structural components shown in FIG. 1 may be altered or replaced with a different structure. In some instances, some of the structural components shown in FIG. 1 may be viewed as optional.

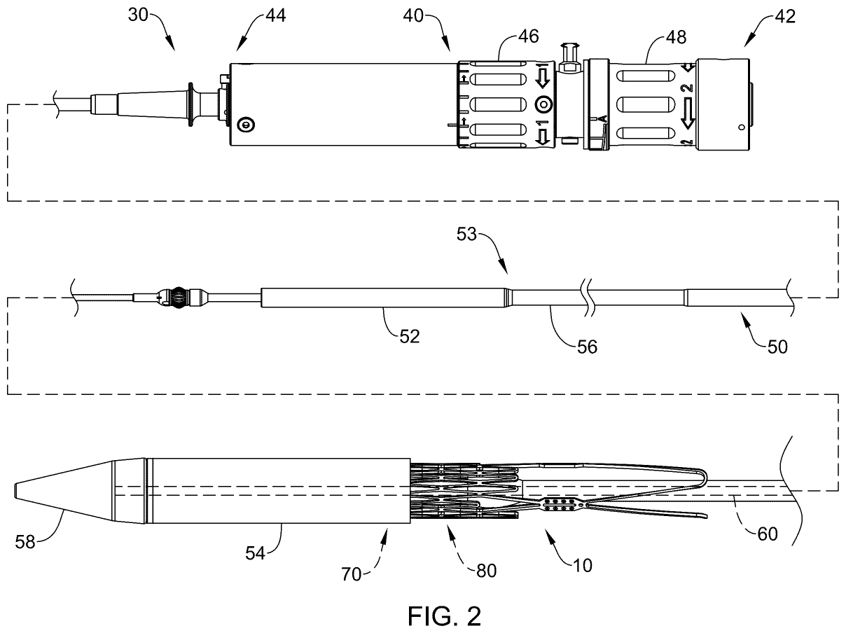

FIG. 2 is a schematic view of an implant delivery system 30 compatible with and/or usable with the replacement heart valve implant 10. It should be noted that FIG. 2 includes at least one change of scale (e.g., all parts of the figure are not drawn to the same scale) to improve viewability and show additional detail of the implant delivery system 30. Additionally, some elements of the replacement heart valve implant 10 are not shown to improve clarity.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob 46 and a second rotatable knob 48. In at least some embodiments, the first rotatable knob 46 and/or the second rotatable knob 48 may be configured to rotate about a central longitudinal axis of the implant delivery system 30 and/or the handle 40.

In some instances, a distal portion of the implant delivery system 30 and/or the elongate shaft assembly 50 may include an implant holding portion configured to engage with and/or retain the replacement heart valve implant 10 and/or the expandable framework 12 in the radially collapsed configuration. The elongate shaft assembly 50 may include an outer tubular member 56 extending distally from the handle 40 and an inner shaft 60 extending distally from the handle 40 within the outer tubular member 56 to a distal tip 58 disposed distal of the implant holding portion. In some instances, the implant holding portion may include a proximal sheath 52 and a distal sheath 54. In some instances, the inner shaft 60 may be slidably disposed within a lumen of the outer tubular member 56. In some instances, the inner shaft 60 may be fixedly attached to the distal sheath 54 and/or the distal tip 58. In some instances, the distal sheath 54 may be fixedly attached to the distal tip 58. In some instances, the distal sheath 54 may extend proximally from the distal tip 58. In some instances, the inner shaft 60 may include and/or at least partially define a guidewire lumen extending therethrough. In some instances, the guidewire lumen may extend through the handle 40. It will be appreciated that there may be a transition 53 between the proximal sheath 52 and the outer tubular member 56. As will be discussed, this transition 53 may be used in locating a proximal loader relative to the implant delivery system 30.

In some instances, the handle 40 may be configured to manipulate and/or translate the proximal sheath 52 and/or the distal sheath 54 relative to each other. In some instances, the first rotatable knob 46 and/or the second rotatable knob 48 may be configured to manipulate and/or axially translate the proximal sheath 52 and/or the distal sheath 54 relative to each other. In some instances, the handle 40 may be configured to manipulate and/or translate the inner shaft 60 relative to the elongate shaft assembly 50 and/or the proximal sheath 52. In some instances, the first rotatable knob 46 and/or the second rotatable knob 48 may be configured to manipulate and/or axially translate the inner shaft 60 relative to the outer tubular member 56 and/or the proximal sheath 52.

During delivery of the replacement heart valve implant 10 to a treatment site, the replacement heart valve implant 10 may be disposed at least partially within the proximal sheath 52 and/or the distal sheath 54 in the radially collapsed configuration. In some embodiments, the proximal sheath 52 and/or the distal sheath 54 may collectively define the implant holding portion of the implant delivery system 30. In some instances, the implant holding portion may be configured to constrain the replacement heart valve implant 10 in the radially collapsed configuration. In some instances, the replacement heart valve implant 10 may be releasably coupled to the inner shaft 60.

In some instances, the proximal sheath 52 may be configured to cover the proximal portion of the replacement heart valve implant 10 in the radially collapsed configuration. As discussed herein, the proximal portion of the replacement heart valve implant 10 may include the inflow end. In some instances, the distal sheath 54 may be configured to cover the distal portion of the replacement heart valve implant 10 in the radially collapsed configuration.

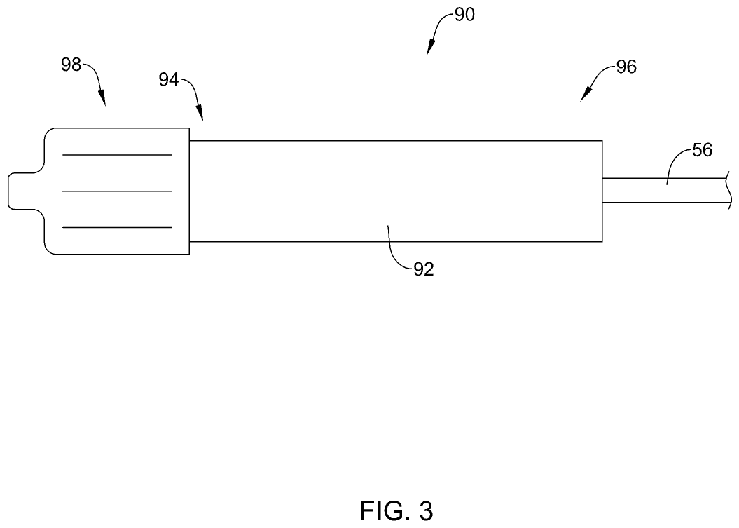

FIG. 3 is a schematic view of an illustrative proximal loader 90 that may be used to facilitate loading a prosthetic valve such as the replacement heart valve implant 10 shown in FIG. 1 into the implant delivery system 30 shown in FIG. 2. The illustrative proximal loader 90 may include an elongate body 92. In some instances, the elongate body 92 may be a clamshell-style body, with a first half hingedly coupled with a second half. This allows the proximal loader 90 to be loaded onto the implant delivery system 30. In some instances, the proximal loader 90 may be loaded onto the implant delivery system 30 such that the proximal loader 90 extends over the transition 93 (FIG. 2) between the proximal sheath 52 and the outer tubular member 56. As a result, the outer tubular member 56 may be seen as extending from within the proximal loader 90.

The proximal loader 90 may be considered as extending from a distal region 94 to a proximal region 96. A pusher cap 98 may be threadedly engaged with a threaded surface (not visible in FIG. 3) that is formed within the distal region 94. The pusher cap 98 may be rotated in a first direction to advance the pusher cap 98 proximally with respect to the proximal loader 90, and may be rotated in an opposing second direction to cause the pusher cap 98 to withdraw distally relative to the proximal loader 90. In some instances, advancing the pusher cap 98 in a proximal direction relative to the proximal loader 90 may urge a prosthetic valve within the pusher cap 98 to move proximally into the proximal loader 90. In some instances, the maximum proximal travel of the pusher cap 98 relative to the proximal loader 90 may be limited by the particular arrangement and positioning of an inner threaded surface within the pusher cap 98 and/or the outer threaded surface on the proximal loader 90. In some instances, there is may be a trade-off between how far into the proximal loader 90 the prosthetic valve is pushed via the pusher cap 98 relative to the position of internal structure within the proximal loader 90 that could potentially damage portions of the prosthetic valve.

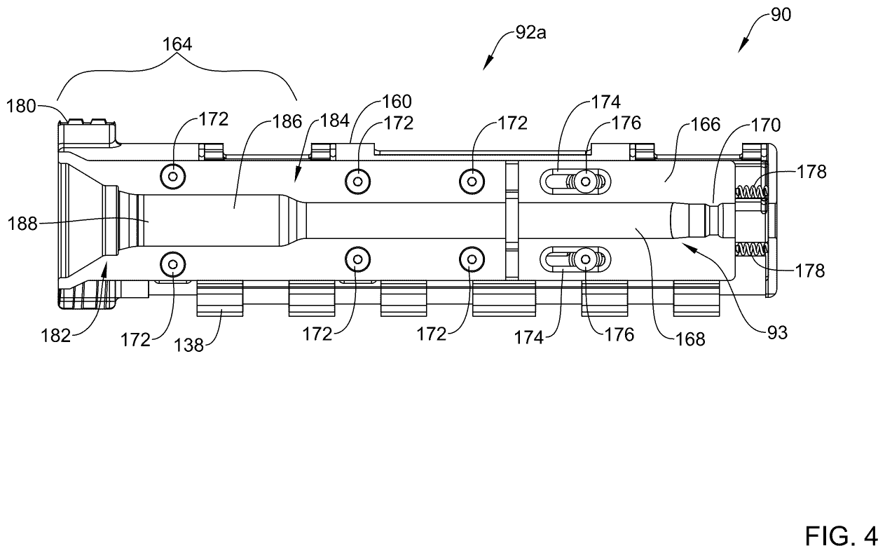

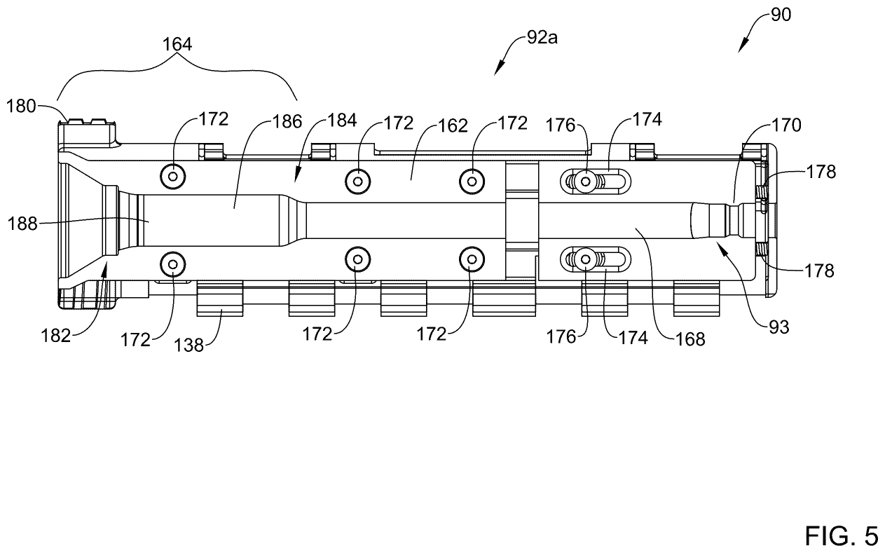

As noted, in some instances, the elongate body 92 of the proximal loader 90 may be a clamshell-style body, with a first half hingedly coupled with a second half. FIG. 4 is a front view of a body half 92a, with a second body half removed for clarity and to illustrate internal components of the body half 92a. The body half 92a includes an outer shell 160. The body half 92a includes a distal member segment 162 that is disposed within the outer shell 160 and that defines half of a valve crimping region 164. The body half 92a includes a proximal member segment 166 that is slidingly disposed within the outer shell 160 and that defines half of a catheter accepting region 168. The catheter accepting region 168 is an elongate void that is dimensioned to allow portions of the proximal sheath 52 and the outer tubular member 56 to extend within the proximal member segment 166. In FIG. 4, the proximal member segment 166 is shown in a position in which the proximal member segment 166 is relatively closer to the distal member segment 162. In some instances, the proximal member segment 166 may be biased into this position. In contrast, FIG. 5 is a front view of the body half 92a, showing the proximal member segment 166 moved into a position in which the proximal member segment 166 is relatively farther from the distal member segment 162. In some instances, the relative position of the proximal member segment 166 shown in FIG. 5 represents the proximal member segment 166 translating against a biasing force that otherwise urges the proximal member segment 166 into the position shown in FIG. 4.

The body half 92a may include hinge components 138 while the second body half (not shown) may include hinge components that are complementary to the hinge components 138. As an example, the hinge components 138 of the body half 92a and the hinge components of a second body half may be adapted to fit together to form a clamshell shape, for example. In some instances, an elongate member (not shown) may extend through the hinge components 138 and the corresponding hinge components of the second body half in order to hold the hinge components 138 and the corresponding hinge components of the second body half together and to allow the body half 92a and the second body half to pivot apart to allow the proximal loader 30 to be clamped over portions of the proximal sheath 52 and the outer tubular member 56. The body half 92a may include one or more latch elements 144 that may be used to secure the body half 92a to the second body half in order to secure the proximal loader 90 in position relative to portions of the proximal sheath 52 and the outer tubular member 56. In some instances, the body half 92a and the second body half are mirror images of each other, apart from possibly laterally offsetting the hinge components 138 from the hinge components of the second body half.

It will be appreciated that because the second body half is a mirror image of the body half 92a, apart from lateral adjustments to the position of the hinge segments 140 relative to the hinge components of the second body half and possibly adjustments to the latches 144 (the second body half may include a latch component complementary to the latches 144), the second body half also includes a distal member segment equivalent to the distal member segment 162 and a proximal member segment equivalent to the proximal member segment 166. The two distal member segments together form the valve crimping region 164 and the two proximal member segments together form the catheter accepting region 168. In some instances, the proximal member segment 166 (and the complementary proximal member segment within the second body half) together form a catheter engaging region 170 that is dimensioned to sufficiently engage the outer tubular member 56 such that applied forces may cause the proximal member segment 166 (and the complementary proximal member segment within the second body half) to translate relative to the distal member segment 162 (and corresponding distal member segment within the second body half).

In some instances, the distal member segment 162 is fixedly secured within the outer shell 160. In some instances, the distal member segment 162 may be adhesively secured in position relative to the outer shell 160. In some instances, the distal member segment 162 may be secured in position via a number of fasteners 172 that extend through apertures formed within the distal member segment 162 and into the outer shell 160. In some instances, the fasteners 172 may be screws having a head adapted to be engaged via a tool and a threaded body that engages the outer shell 160. As shown, the fasteners 172 have a hex-shaped recess adapted to receive an Allen wrench, but other styles are also contemplated. The fasteners 172 may have a recess formed to receive a standard screwdriver or a Phillips screwdriver, for example. The corresponding distal member segment within the second body half may be secured in a similar manner. In some instances, the distal member segment 162 may be considered as being a distal insert that is separately created and then secured in position within the body half 92a.

In some instances, the proximal member segment 166 is slidingly secured within the outer shell 160. In some instances, the proximal member segment 166 may include one or more mounting slots 174 that extend through the proximal member segment 166. A length of each of the mounting slots 174 defines how far the proximal member segment 166 is permitted to translate relative to the outer shell 160 (and thus relative to the distal member segment 162). In some instances, the proximal member segment 166 may be slidingly secured in position via fasteners 176 that extend through each of the mounting slots 174 and into the outer shell 160. As shown, the fasteners 176 have a hex-shaped recess adapted to receive an Allen wrench, but other styles are also contemplated. The fasteners 176 may have a recess formed to receive a standard screwdriver or a Phillips screwdriver, for example. The corresponding proximal member segment within the second body half may be slidingly secured in a similar manner. In some instances, the proximal member segment 166 may be considered as being a proximal insert that is separately created and then slidingly secured in position within the body half 92a. In FIG. 4, which shows the proximal member segment 166 positioned relatively closer to the distal member segment 162, the fasteners 176 are positioned closer to a right hand side of the mounting slots 174. In contrast, in FIG. 5, which shows the proximal member segment 166 positioned relatively farther away from the distal member segment 162, the fasteners 176 can be seen as being positioned closer to a left hand side of the mounting slots 174. Obviously, the fasteners 176 themselves are not moving, but the position of the fasteners 176 within the mounting slots 174 changes as the proximal member segment 166 moves relative to the fasteners 176.

In some instances, the proximal member segment 166 may be biased into the position shown in FIG. 4. In some instances, the body half 92a includes one or more biasing members that bias the proximal member segment 166 towards the distal member segment 162. As shown, a pair of springs 178 are positioned between the proximal member segment 166 and the outer shell 160. The proximal member 166 may be able to translate in a direction away from the distal member segment 162 by compressing the springs 178, for example. Relative forces exerted between the proximal loader 90 and the outer tubular member 56 may cause the proximal member segment 166 (and the corresponding proximal member segment within the second body half) to move relative to the distal member segment 162 as a result of the interaction between the outer tubular member 56 and the catheter engaging region 170 of the proximal member segment 166 (and the corresponding proximal member segment within the second body half). To illustrate, FIG. 5 shows the proximal member segment 166 moved away from the distal member segment 162, against the biasing force applied by the springs 178. As can be seen in FIG. 5, the springs 178 have been compressed from the relaxed configuration of the springs 178 shown in FIG. 4.

In some instances, the outer shell 160 includes an outer threaded surface 180 that is adapted to engage a corresponding inner threaded surface within the pusher cap 98. In some instances, the outer shell forming part of the second body half also includes a corresponding outer threaded surface such that the outer threaded surface on the outer shell forming part of the second body half in combination with the outer threaded surface 180 of the outer shell 160 forming part of the body half 92a forms several complete threads that extend around a periphery of the proximal loader 90 in a spiral or helical manner. In some instances, the outer threaded surface 180 and the corresponding outer threaded surface on the outer shell forming part of the second body half together may be modified to limit relative axial travel of the pusher cap 98 relative to the proximal loader 90 in order to reduce or eliminate possible damage to the prosthetic valve. In some instances, the inner threaded surface of the pusher cap 98 may additionally or alternatively be modified to limit relative axial travel of the pusher cap 98 relative to the proximal loader 90 in order to reduce or eliminate possible damage to the prosthetic valve.

The valve crimping region 164 includes a first tapered portion 182, a second tapered portion 184 and a constant diameter portion 186 extending between the first tapered portion 182 and the second tapered portion 184. In some instances, contact between the prosthetic valve and the second tapered portion 184 may be problematic, possible bending or dislodging one or more of the struts forming the prosthetic valve such as the replacement heart valve implant 10. In some instances, relative movement between the distal member segment 162 and the proximal member segment 166 (and the corresponding distal member segment and the corresponding proximal member segment of the second body half), in combination with particular feature of the pusher cap 98, may help to reduce or eliminate contact between the prosthetic valve and the second tapered portion 186.

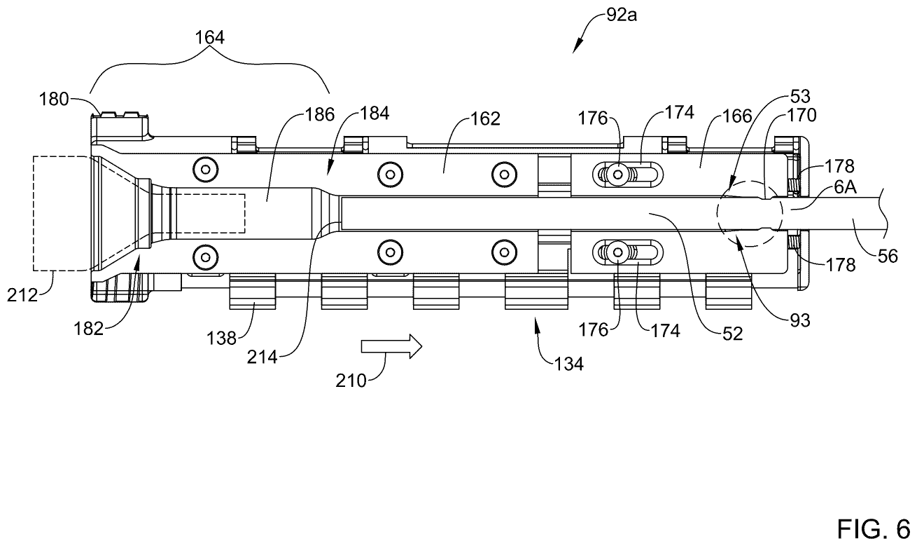

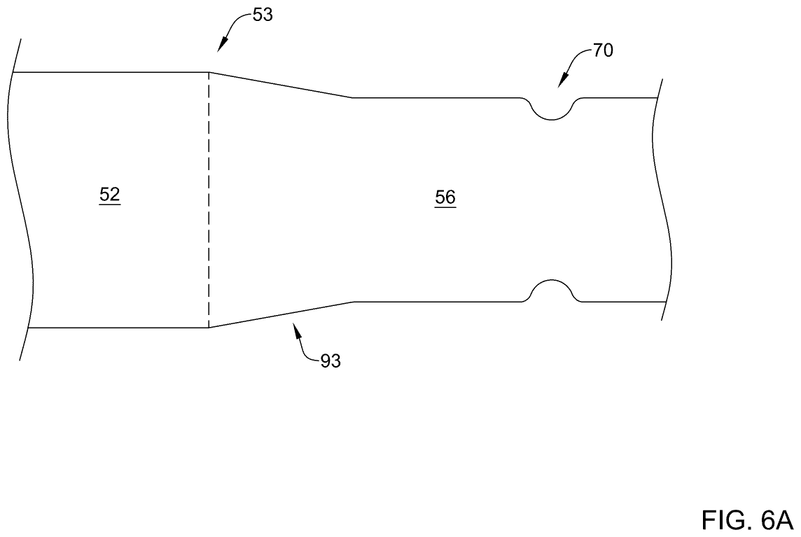

FIGS. 6 through 8 show parts of a four step loading process. Step 1 of the four step loading process may be seen in FIG. 4, which shows the springs 178 pushing the proximal member segment 166 towards the distal member segment 162. Because the catheter engaging region 170 engages the outer tubular member 56, the springs 178 may be considered as pushing the outer tubular member 56 distally. Step 2 is shown in FIG. 6. The proximal sheath 52 and the outer tubular member 56 are introduced into the assembly by pulling it in the direction of an arrow 210. This motion compresses the springs 178. A valve 212, generally representative of the replacement heart valve implant 10 shown in FIG. 1, and more particularly representative of the outflow end of the replacement heart valve implant 10, including the stabilization arches 18, is partially loaded into the stent crimping region 164 of the distal member segment 162.

FIG. 6A shows an enlarged portion of FIG. 6. Specifically, FIG. 6 shows that the passage extending through the proximal segment 166 includes a transition 93 in which the diameter of the passage decreases. This transition 93 in the diameter of the passage corresponds to the transition 53, which as noted is the transition between the proximal sheath 52 and the outer tubular member 56. The proximal sheath 52 extends distally from the transition 53 and the outer tubular member 56 extends proximally from the transition 53. When the proximal loader 90 is loaded onto the implant delivery system 30, the transition 53 within the implant delivery system 30 is targeted to be aligned with the transition 93 within the passage of the proximal member segment 166.

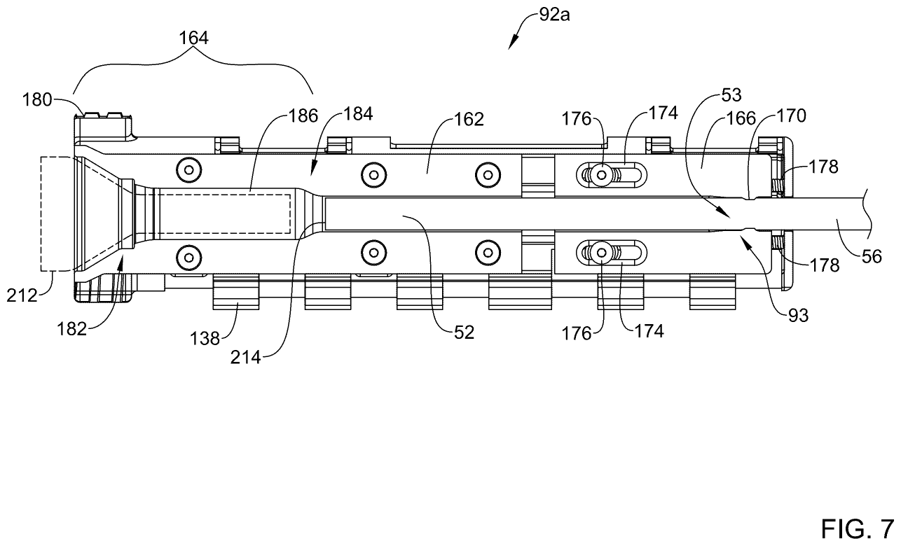

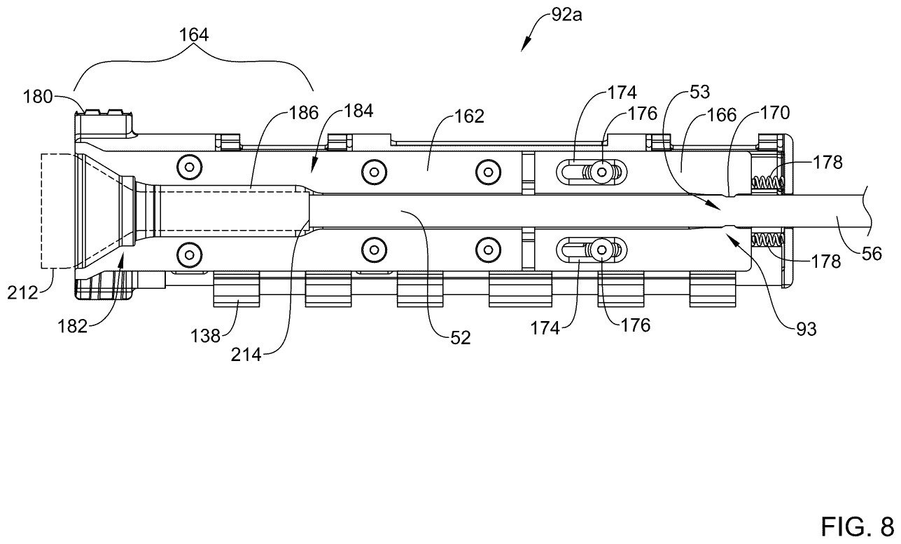

Step 3 is shown in FIG. 7. The valve 212 is crimped by applying compression via the pusher cap 98. This distal compression means that the springs 178 remain compressed, as shown. The distal movement will be limited by the pusher cap 98 being adapted to prevent the valve 212 from being pushed into contact with the second tapered region 184. At this point, a distal end 214 of the proximal sheath 52 remains proximal of the second tapered region 184. Step 4, which is the completion of proximal loading, is shown in FIG. 8. Once the user bottoms out the pusher cap 98, as a result of the compressed springs 178, the proximal sheath 52 moves distally such that the distal end 214 of the proximal sheath 52 moves into the second tapered region 184. As a result, the valve 212 is prevented from contacting the second tapered region 184, thereby limiting or even preventing possible damage to the valve 212.

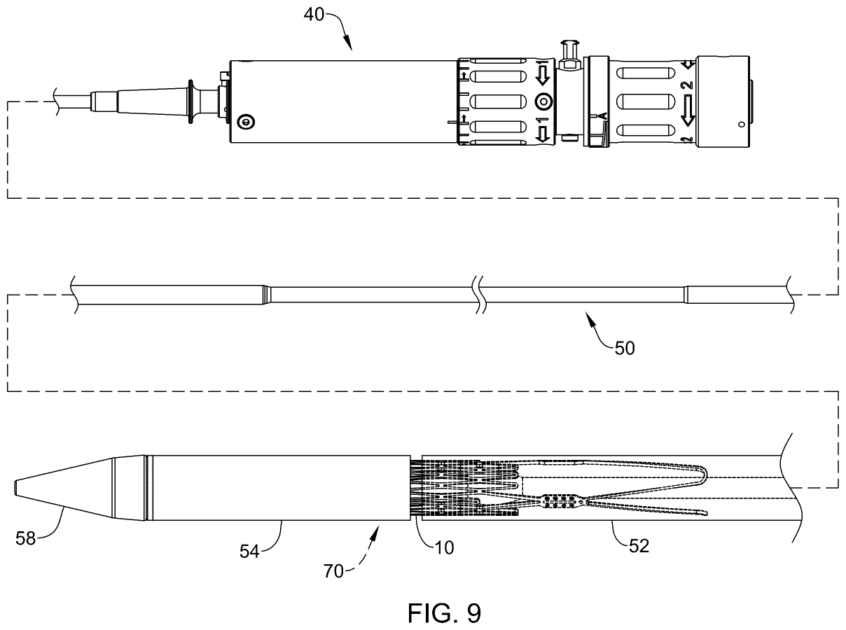

In some instances, loading the replacement heart valve implant 10 into the plant delivery system 30 may include translating the proximal sheath 52 of the implant delivery system 30 over the proximal portion of the replacement heart valve implant 10, as seen in FIG. 9. In some instances, the method may include translating the proximal sheath 52 of the implant delivery system 30 distally over the proximal portion of the replacement heart valve implant 10. In some instances, the method may include translating the proximal sheath 52 of the implant delivery system 30 distally relative to the inner shaft 60 and/or the stent holder 70.

In some instances, the replacement heart valve implant 10 and/or the expandable framework 12 may be retained in the radially collapsed configuration by the proximal sheath 52 and the distal sheath 54 in a delivery configuration of the implant delivery system 30. In some instances, the proximal sheath 52 may be disposed adjacent to the distal sheath 54 in the delivery configuration. In some instances, the proximal sheath 52 may abut the distal sheath 54 in the delivery configuration. In some instances, the proximal sheath 52 may be axially spaced apart from the distal sheath 54 in the delivery configuration. In some instances, the proximal sheath 52 may be axially spaced apart from the distal sheath 54 in the delivery configuration by less than 20% of an overall length of the replacement heart valve implant 10 and/or the expandable framework 12. In some instances, the proximal sheath 52 may be axially spaced apart from the distal sheath 54 in the delivery configuration by less than 15% of an overall length of the replacement heart valve implant 10 and/or the expandable framework 12. In some instances, the proximal sheath 52 may be axially spaced apart from the distal sheath 54 in the delivery configuration by less than 10% of an overall length of the replacement heart valve implant 10 and/or the expandable framework 12. In some instances, the proximal sheath 52 may be axially spaced apart from the distal sheath 54 in the delivery configuration by less than 5% of an overall length of the replacement heart valve implant 10 and/or the expandable framework 12.

The components, features, and/or methods described herein may simplify loading of the replacement heart valve implant 10 into the replacement heart valve system and/or the implant delivery system 30. Simplified loading may reduce training requirements for physicians and/or hospital staff using and/or preparing the replacement heart valve system. Simplified loading may reduce damage to the replacement heart valve system and/or components thereof. Simplified loading may reduce scrap rates and/or cost due to incorrect loading of the replacement heart valve implant 10. The components, features, and/or methods described herein may reduce cost to manufacture the replacement heart valve system compared to existing systems and/or products. Other benefits are also contemplated and/or expected.

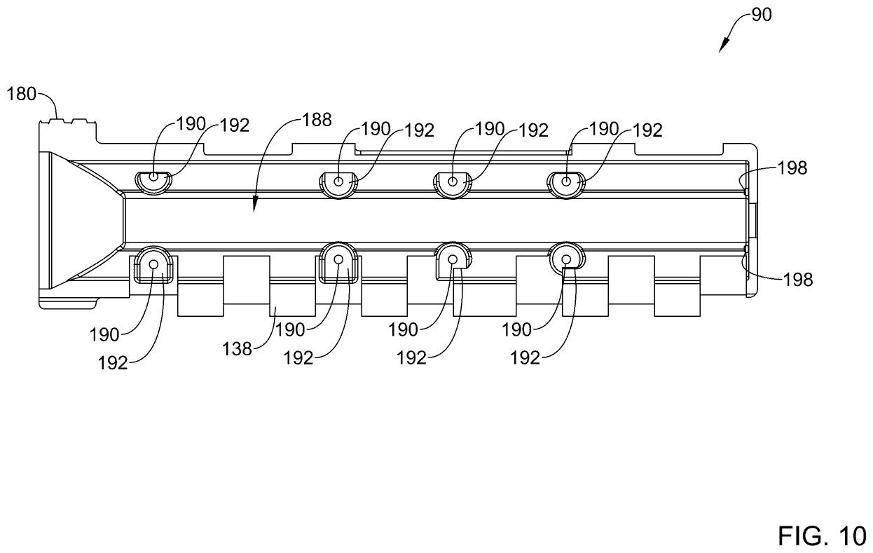

In some instances, the distal member segment 162 and the proximal member segment 166 are separately formed and the distal member segment 162 is secured to the outer shell 160 and the proximal member segment 166 is slidingly secured to the outer shell 160. FIG. 10 is a front view of the outer shell 160. The second body half may be considered as including an outer shell that is essentially the same as the outer shell 160, apart from lateral changes in where the hinge components of the second body half are positioned (to mesh with the hinge components 138). The outer shell 160 defines a void 188 that is adapted to accommodate the distal member segment 162 and the proximal member segment 166 therein. In some instances, the outer shell 160 includes a number of apertures 190 that are adapted to receive a threaded body portion of the fasteners 172 and 176, respectively. In some instances, the apertures 190 may be threaded prior to insertion of the fasteners 172 and 176. In some instances, the apertures 190 may not be threaded prior to insertion of the fasteners 172 and 176, and instead, the fasteners 172 and 176 themselves may cut threads into the apertures 190 as the fasteners 172 and 176 are secured in place. In some instances, the apertures 1 90 may each be disposed within a flat spot 192 that helps in aligning the fasteners 172 and 176 with the apertures 190. In some instances, the outer shell 160 may include spring mounting pegs 198 that help to locate and secure the springs 178 relative to the outer shell 160.

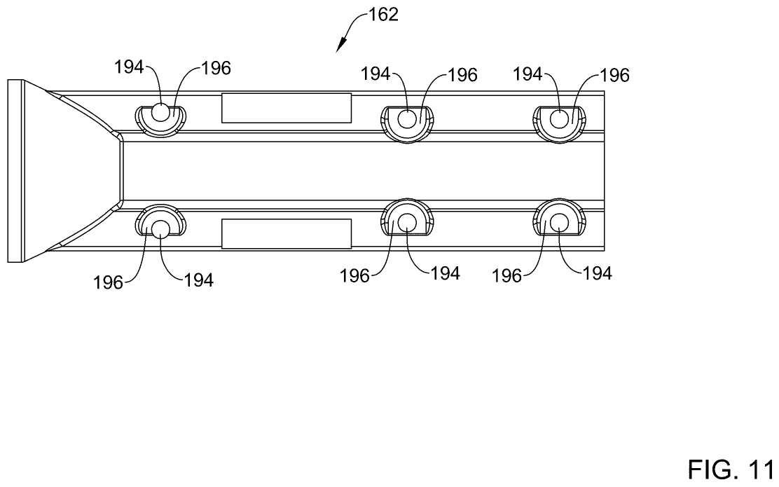

FIG. 11 is a back view of the distal member segment 162, showing a profile that fits into the void 188 formed in the outer shell 160. The back side of the distal member 162 includes a number of apertures 194 that are adapted to allow the fasteners 172 to extend therethrough. The fasteners 172 extend freely through the apertures 194, and thus are not threadedly engaged with the apertures 194. In some instances, the apertures 194 extending through the distal member segment 162 may be larger in diameter than the apertures 190 formed in the outer shell 160 that the threaded body portions of the fasteners 172 engage. In some instances, as shown, the apertures 194 may be formed within flat spots 196 formed on the back of the distal member segment 162 to align with the corresponding flat spots 192 formed on the outer shell 160.

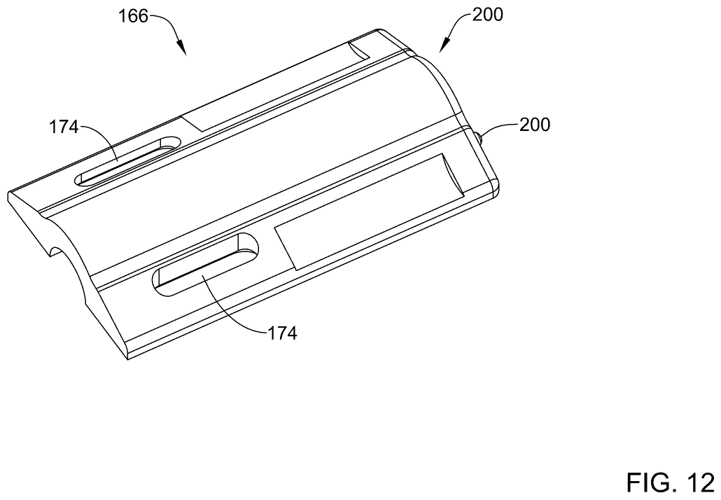

FIG. 12 is a back view of the proximal member segment 166, showing a profile that fits into the void 188 formed in the outer shell 160. As can be seen, the mounting slots 174 extend axially within the proximal member segment 166. In some instances, the proximal member segment 166 may include spring mounting pegs 198 that help to locate and secure the springs 178 relative to the proximal member segment 166.

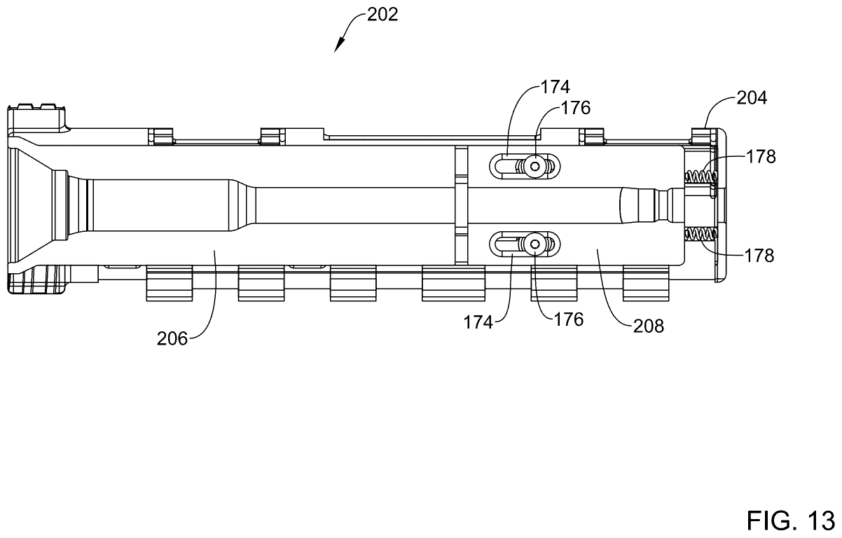

As shown in FIGS. 10 through 12, the distal member segment 62 is separately formed and then subsequently secured relative to the outer shell 60. FIG. 13 is a front view of a first body half 202 that includes an outer shell 204. The first body half 202 includes a distal member segment 206 that is integrally formed with the outer shell 204. As an example, the distal member segment 206 may be molded as a single component as part of the outer shell 204. The proximal member segment 208 may be separately formed and then slidingly secured to the outer shell 204, much like the proximal member segment 166 already described.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A proximal loader, comprising:
an elongate body releasably securable about a catheter, the elongate body including a first body half hingedly coupled with a second body half;
each of the first body half and the second body half including:
an outer shell;
a distal member segment disposed within the outer shell and defining half of a valve crimping region;
a proximal member segment slidingly disposed within the outer shell and defining half of a catheter accepting region.

2. The proximal loader of claim 1, wherein each distal member segment is fixedly secured to the corresponding outer shell.

3. The proximal loader of claim 2, wherein each distal member segment comprises a distal insert that is fastened to the outer shell.

4. The proximal loader of claim 1, wherein each distal member segment is integrally molded as part of the corresponding outer shell.

5. The proximal loader of claim 1, wherein each proximal member segment is slidingly secured to the corresponding outer shell.

6. The proximal loader of claim 1, wherein each of the first body half and the second body half further comprise one or more biasing members extending between the proximal member segment and the corresponding outer shell.

7. The proximal loader of claim 6, wherein the one or more biasing members comprise springs.

8. The proximal loader of claim 1, wherein each proximal member segment comprises a proximal insert having two or more elongate mounting slots formed within the proximal insert, and the proximal insert is slidingly secured to the corresponding outer shell via fasteners extending through each of the two or more elongate mounting slots and engaging in the corresponding outer shell.

9. The proximal loader of claim 1, wherein the first body half and the second body half each include a semicircular outer threaded surface adapted to threadedly engage a pusher cap that is used to advance the prosthetic valve proximally into the catheter.

10. The proximal loader of claim 1, wherein each proximal member segment further defines a catheter engaging region that is adapted to secure the proximal member relative to the catheter.

11. A proximal loader, comprising:
an outer shell;
a distal member disposed within the outer shell, the distal member defining a valve crimping region;
a proximal member slidingly disposed within the outer shell, the proximal member defining a catheter accepting region;
the proximal member further defining a catheter engaging region that is adapted to secure the proximal member relative to the catheter;
wherein the proximal loader is adapted to be hingedly opened in order to secure the proximal loader about the catheter.

12. The proximal loader of claim 11, wherein the outer shell comprises a first shell half and a second shell half.

13. The proximal loader of claim 12, wherein the distal member comprises:
a first distal member segment disposed within the first shell half; and
a second distal member segment disposed within the second shell half.

14. The proximal loader of claim 13, wherein the first distal member segment is fixedly secured within the first shell half and the second distal member segment is fixedly secured within the second shell half.

15. The proximal loader of claim 12, wherein the proximal member comprises:
a first proximal member segment slidingly secured within the first shell half; and
a second proximal member segment slidingly secured within the second shell half.

16. The proximal loader of claim 15, wherein:
the first proximal member is slidingly secured within the first shell half via one or more fasteners extending through the first proximal member and into the first shell half; and
the second proximal member is slidingly secured within the second shell half via one or more fasteners extending through the second proximal member and into the second shell half.

17. The proximal loader of claim 11, further comprising one or more biasing members extending between the outer shell and the proximal member.

18. A proximal loader, comprising:
a first outer shell;
a first distal member segment disposed within the first outer shell;
a first proximal member segment slidingly disposed within the first outer shell;
a first biasing member disposed between the first proximal member segment and the first outer shell;
a second outer shell hingedly secured to the first outer shell;
a second distal member segment disposed within the second outer shell, the first distal member and the second distal member together defining a valve crimping region;
a second proximal member segment slidingly disposed within the second outer shell, the first proximal member segment and the second proximal member segment together defining a catheter accepting region; and
a second biasing member disposed between the second proximal member segment and the second outer shell.

19. The proximal loader of claim 18, wherein:
the first biasing member biases the first proximal member segment towards the first distal member segment; and
the second biasing member biases the second proximal member segment towards the second distal member segment.

20. The proximal loader of claim 18, wherein the first proximal member segment and the second proximal member segment together define a catheter engaging region that is adapted to secure a catheter extending between the first proximal member segment and the second proximal member segment against movement relative to the first proximal member segment and the second proximal member segment.
